(19) United States
(12) Patent Application Publication
(10) Pub. No.: US 2013/0315987 A1
(43) Pub. Date: Nov. 28, 2013

(54) LYOPHILIZED LIPOSOME COMPOSITION ENCAPSULATING A WATER-SOLUBLE DRUG AND PREPARATION PROCESS THEREOF

(71) Applicant: Zhijun Lu, Guangdong (CN)

(72) Inventor: Zhijun Lu, Guangdong (CN)

(73) Assignee: REGENEX CORPORATION, Guangdong (CN)

(21) Appl. No.: 13/990,354

(22) PCT Filed: Nov. 25, 2011
(86) PCT No.: PCT/CN2011/082958

§ 371 Date: Aug. 12, 2013

(30) Foreign Application Priority Data
Nov. 29, 2010 (CN) 201010562307.2

(51) Int. Cl. A61K 47/40; A61K 9/127; A61K 47/26

(52) U.S. Cl. CPC A61K 47/40; A61K 47/26; A61K 9/127
USPC 424/450; 514/777; 514/34

(57) ABSTRACT

Disclosed is a lyophilized liposome composition encapsulating a water-soluble drug and a preparation process thereof. The lyophilized liposome composition comprises a water-soluble drug, a phospholipid, a polyethylene glycol-derivatized phospholipid, cholesterol and a lyoprotectant, wherein the lyoprotectant comprises a saccharide and a cyclodextrin or cyclodextrin derivative. The encapsulation rate of the lyophilized liposome composition encapsulating the water-soluble drug is ≧90%.

### FIELD OF THE INVENTION

The present invention relates to the medical field, and particularly to a lyophilized liposome composition encapsulating a water-soluble drug and a preparation process thereof.

### BACKGROUND OF THE INVENTION

Liposomes are bilayer vesicles comprised of phospholipids. Liposomes can be classified into unilamellar vesicles and multilamellar vesicles. Their structures have been disclosed in numerous literatures and monographs. Liposomes, as a vehicle for drug delivery, meet many requirements for treating diseases when using drug formulations. Liposomes are advantageous as they can encapsulate drugs and deliver said drugs to the focus of the disease; administration of a drug encapsulated in liposomes enable greater control over drug distribution within the body, lower the plasma concentration of the encapsulated drug, and subsequently, lower the toxicity.

There are plenty of methods for the preparation of liposomes, as described by literatures and monographs alike, such as Szoka and Papahadjopoulos, Ann Re. Biophysics Bioeng. 9:467-508 (1980), Liposome Technology, Preparation of Liposome, Vol I, Gregoriadis(Ed), CRC Press, Inc. (1984), and [symbol] New dosage forms and technologies in modern medicine [symbol] . Liposome encapsulation technologies have been also disclosed by several patent literatures, such as U.S. Pat. No. 4,235,871 and U.S. Pat. No. 4,016,100.

As shown in the table below, liposome technology has been successfully applied to encapsulation of several pharmaceutical drugs, which realized industrialized production of some liposomal drugs (the table lists liposomal drug products currently approved for sale). Specifically, U.S. Pat. No. 5,213,804 described a liposomal composite which was successfully applied to encapsulation of anthracycline drugs such as doxorubicin hydrochloride. The patent contributes to the production of Doxil, a liposome-encapsulated doxorubicin injection, which is now commercially available to treat AIDS, tumors and so on, wherein it exhibits an improved therapeutic effect and decreased toxicity due to the targeting properties of liposomes.

TABLE
Summary of currently approved liposomal drug products

| | | | | Approved | | |
|---|---|---|---|---|---|---|
| | | | | countries(or | | |
| | | | | regions) | | |
| Active | | | | and date of | Shelf | |
| ingredient | Trade name | Manufacturer | Indications | release | life | Remarks |
| Doxorubicin | Doxil | Sequus | AIDS, | Europe and | 20 | Water-soluble |
| Hydrochloride | | | cancer | America | months | drug, liquid |
| | | | | 1995 | | formulation |
| Daunorubicin | Daunoxome | Nexstar | AIDS, | Europe and | 18 | Water-soluble |
| Citrate | | | cancer | America | months | drug, liquid |
| | | | | 1996 | | formulation |
| Cytarabine | Depocyt | Depotech | Acute | Released in | 18 | Water-soluble |
| | | | Myeloid | 1999 | months | drug, liquid |
| | | | Leukaemia | | | formulation |
| Amphotericin | Ambiosome | NeXstar | Fungal | Released in | 36 | Water-insoluble |
| B | | Pharmaceuticals | infection | 1990 | months | drug, |
| | | | | | | lyophilized |
| | | | | | | formulation |

A majority of the currently approved liposomal drug products are liquid formulations. However, liposomes are prone to aggregation, fusion, phospholipid hydrolysis, and drug leakage in an aqueous medium, consequently shortening shelf life. In particular, the interaction between water-soluble drugs and liposome membranes is relatively weak, which further affects the long-term stability of the drug. As listed above, a majority of the liposomal drug products have a shelf life of 18-20 months. Actually, there are numerous inconveniences in logistical distribution and usage of a pharmaceutical product if it has a shelf life lesser than 2 years. Therefore, although there is a plenty of researches on liquid formulation of liposomal drugs, the commercially available and widely used products are only the small subset mentioned above.

Extensive researches and attempts have been made to improve the shelf life of liposomal drugs to enhance its practical uses. In 1978, Vanleberghe first reported increasing storage stability of liposomal drugs by using a lyophilization method. In 1990, researchers discovered the application of lyophilization improved the stability of liposomes encapsulating water-insoluble drugs, such as amphotericin B. This technology has since been successfully applied to production of a liposomal amphotericin B (Trade name: Ambiosome) with a shelf life as long as 3 years, which was launched in 1990.

Since then, researchers have attempted to encapsulate other drugs using liposomes followed by a lyophilization process. However, it was observed during the freezing process that complications such as formation of ice crystals, changes in osmotic pressure, phase separation, phase variation and phase transition all could cause liposome membranes to fold, merge, be damaged or experience drug leakage. The drug leakage problem is particularly prominent in water-soluble drug, due to its weak interaction with liposomal membrane, and as such, making it much more difficult to utilize the lyophilization method. No existing water-soluble drug has been developed into a lyophilized liposome composition capable of being stored for a long term (shelf life ≧18 months).

To resolve the problems of poor long-term stability for liposome suspensions, and to solve the problems of low encapsulation rate and drug leakage resulted from the freeze-dry process, it was found that adding a suitable quantity of lyoprotectant prior to the freeze-dry process could prevent complications such as leakage of core material from liposome capsule, and aggregation and fusion between particles. The addition also transforms the liposome from an unilamellar vesicle to a multilamellar vesicle. Accordingly, screening for suitable lyoprotectants, and utilization of lyophilization to realize the long-term storage stability of liposomes, had been the main focus of liposome-related researches for nearly twenty years. Researchers are continuing their ongoing efforts and researches to overcome obstacles in the liposome lyophilization process, and are screening for suitable lyoprotectants. U.S. Pat. No. 431,172 disclosed a type of lyophilized liposome, which is consisted of amphipathic phospholipids and at least one lipid soluble drug capable of being dissolved in organic solvents. According to the specification of the patent, the encapsulation rate after redissolution of the lyophilized product is only 80%.

Nonetheless, it has never been reported in published documents that the use of existing lyoprotectants are capable of reaching an encapsulation rate 90% and a shelf life over 20 months in a lyophilized liposome encapsulating a water-soluble drug.

### SUMMARY OF THE INVENTION

An object of this invention is to provide a lyoprotectant composition used for the preparation of a liposomal drug formulation, which comprises a saccharide and a cyclodextrin or cyclodextrin derivative, said lyoprotectant composition is particularly suitable for the preparation of a liposome encapsulating a water-soluble drug. In the lyoprotectant composition of the present invention, the mass ratio of the saccharide to the cyclodextrin or the cyclodextrin derivative is 50-90:5-35, preferably, the mass ratio is 70-80:5-15. The lyoprotectant composition of the present invention can be prepared by mixing the saccharide with the cyclodextrin or the cyclodextrin derivative.

Another object of the present invention is to provide a lyophilized liposome composition encapsulating a water-soluble drug, so as to overcome the difficulties such as the inability to store liposomal suspensions of water-soluble drugs in a stable and long-term manner. The present invention further resolves difficulties such as particle aggregation, particle fusion, and low encapsulation rate during the lyophilization process. The present invention also provides a stable lyophilized liposome encapsulating a water-soluble drug with a shelf life of ≧24 months and an encapsulation rate of 90%. A process for preparing the lyophilized liposome is also provided.

The lyophilized liposome composition of the present invention is achieved through the following technical solutions. A lyophilized liposome composition encapsulating a water-soluble drug comprises the following ingredients: a water-soluble drug, a phospholipid, a polyethylene glycol-derivatized phospholipid, cholesterol, a saccharide and a cyclodextrin or cyclodextrin derivative.

Preferably, the weight percent of each ingredient contained in the lyophilized liposome composition is as follows: 0.5-10% by weigh of the water-soluble drug, 1-10% by weigh of the phospholipid, 1-12% by weigh of the polyethylene glycol-derivatized phospholipid, 1-15% by weight of cholesterol, 50-90% by weight of the saccharide, and 5-35% by weight of the cyclodextrin or cyclodextrin derivative.

In a preferred embodiment of the present invention, the weight percent of each ingredient contained in the lyophilized liposome composition is as follows: 0.5-10% by weight of the water-soluble drug, 1-10% by weight of the phospholipid, 1-12% by weight of the polyethylene glycol-derivatized phospholipid, 1-15% by weight of cholesterol, 70-80% by weight of the saccharide, and 5-15% by weight of the cyclodextrin or cyclodextrin derivative.

The saccharide used in the present invention is one or more selected from the group comprising trehalose, xylitol, glucose, galactose, mannitol, maltose, sucrose, lactose and fructose. Preferably, the saccharide is one or more selected from the group consisting of D-glucose, lactose, D-mannitol, maltose and sucrose. Preferably, the saccharide can perform a lyoprotective function when added to outer phase of liposome dispersion solutions containing either empty-liposomes or liposomes encapsulating a water-soluble drug during the preparation of the lyophilized liposome composition of the present invention.

Preferably, the saccharide is lactose and/or sucrose.

The cyclodextrin or the cyclodextrin derivative used in the present invention is one or more selected from the group comprising α-cyclodextrin, β-cyclodextrin, γ-cyclodextrin, hydroxypropyl-α-cyclodextrin, hydroxypropyl-β-cyclodextrin, 2-hydroxypropyl-beta-cyclodextrin, hydroxypropyl-γ-cyclodextrin, hydroxyethyl-α-cyclodextrin, hydroxyethyl-3-cyclodextrin, 2-hydroxyethyl-β-cyclodextrin, hydroxyethyl-γ-cyclodextrin. Preferably, the cyclodextrin or cyclodextrin derivative can perform a lyoprotective function when added to outer phase of liposome dispersion solutions containing liposomes encapsulating a water-soluble drug during the preparation of the lyophilized liposome composition of the present invention.

Preferably, the cyclodextrin or the cyclodextrin derivative is one or more selected from the group consisting of hydroxypropyl-α-cyclodextrin, hydroxypropyl-β-cyclodextrin, 2-hydroxypropyl-β-cyclodextrin and hydroxypropyl-γ-cyclodextrin.

More preferably, the cyclodextrin or the cyclodextrin derivative is either hydroxypropyl-β-cyclodextrin or 2-hydroxypropyl-β-cyclodextrin.

The phospholipid of the present invention is selected from the group consisting of egg lecithin, soya bean lecithin, distearoylphosphatidylglycerol (DSPG), hydrogenated soya phosphatidyl choline (HSPC), dioleoylphosphatidylcholine (DOPC), dipalmitoylphosphatidylglycerol (DPPG) and distearoylphosphatidylethanolamine (DSPE).

The portion of phospholipid in the polyethylene glycol-derivatized phospholipid is one selected from the group consisting of soya bean lecithin, distearoyl phosphatidylglycerol (DSPG), hydrogenated soya phosphatidyl choline (HSPC), dioleoylphosphatidylcholine (DOPC), dipalmitoylphosphatidylglycerol (DPPG) and distearoylphosphatidylethanolamine (DSPE). Preferably, the polyethylene glycol of the polyethylene glycol-derivatized phospholipid has a molecular weight in the range from 2000 to 4000. More preferably, the polyethylene glycol of the polyethylene glycol-derivatized phospholipid has a molecular weight of 2000. For example, DSPE-mPEG2000, DPPG-mPEG2000, HSPC-mPEG2000 and DOPC-mPEG2000 are used in the present invention.

The water-soluble drug used in the present invention is selected from the group consisting of anti-tumor drugs, anti-infective drugs and hormone drugs. Preferably, when an anti-tumor drug is used as the water-soluble drug, the anti-tumor drug is one or more selected from the group consisting of doxorubicin, daunorubicin, pirarubicin, epirubicin, and pharmaceutically acceptable salts thereof.

Optionally, the lyophilized liposome composition encapsulating a water-soluble drug can further include a certain amount of a supplement such as histidine, glycine, glutamic acid, etc.

In a preferred embodiment of the present invention, the lyophilized liposome composition encapsulating a water-soluble drug comprises: 1-3% by weight of doxorubicin hydrochloride, 5-8% by weight of hydrogenated soya phosphatidylcholine (HSPC), 2-7% by weight of DSPE-mPEG2000 as the polyethylene glycol-derivatized phospholipid, 1-7% by weight of cholesterol, 70-80% by weight of sucrose and 5-15% by weight of hydroxypropyl-β-cyclodextrin.

In another embodiment of the present invention, the lyophilized liposome composition encapsulating water-soluble drugs comprises: 1-3% by weight of daunorubicin hydrochloride, 5-8% by weight of hydrogenated soya phosphatidylcholine (HSPC), 2-7% by weight of the DSPE-mPEG2000 as the polyethylene glycol-derivatized phospholipid, 1-7% by weight of cholesterol, 70-80% by weight of sucrose and 5-15% by weight of hydroxypropyl-β-cyclodextrin.

The present invention also provides a process of preparing a lyophilized liposome composition encapsulating a water-soluble drug, comprising the steps of: dissolving a phospholipid, a polyethylene glycol-derivatized phospholipid, and cholesterol in an organic solvent to obtain a clear solution A; adding a first buffer solution into the clear solution A with continuous stirring under a 50-80° C. water bath, extruding the resultant product to obtain empty-liposomes D with an average particle diameter from approximately 10 to 500 nm; dialyzing the first buffer solution from the empty-liposomes D by adding a second buffer solution to obtain empty-liposomes E; dissolving the water-soluble drug and the saccharide as a lyoprotectant in the second buffer to obtain a solution B; mixing the empty-liposomes E and the solution B, encapsulating at a temperature between 40-100° C. for 5-60 minutes followed by cooling at room temperature to obtain liposomes F, which encapsulating the water-soluble drug in its inner phase; preparing aliquots of the liposomes F after adding the cyclodextrin or cyclodextrin derivative as a lyoprotectant; lyophilizing the aliquots to obtain the lyophilized liposome composition encapsulating the water-soluble drug.

The present invention provides another process of preparing a lyophilized liposome composition encapsulating a water-soluble drug, comprising the steps of: dissolving a phospholipid, a polyethylene glycol-derivatized phospholipid and cholesterol in an organic solvent to obtain a clear solution A; adding a first buffer solution into the clear solution A with continuous stirring under a 50-80° C. water bath, extruding the resultant product to obtain empty-liposomes D with an average particle diameter from approximately 10 to 500 nm; dialyzing the first buffer solution from the empty-liposomes D by adding a second buffer solution to obtain empty-liposomes E; dissolving the water-soluble drug in the second buffer to obtain a solution B; mixing the empty-liposomes E and the solution B, encapsulating at a temperature between 40-100° C. for 5-60 minutes followed by cooling at room temperature to obtain liposomes F, with the inner phase of the liposomes F encapsulating the water-soluble drug; preparing aliquots of the liposomes F after adding the saccharide and the cyclodextrin or cyclodextrin derivative as a combined lyoprotectant; lyophilizing the aliquots to obtain the lyophilized liposome composition encapsulating the water-soluble drug.

As used herein, the “outer phase of liposome” is the whole space between individual liposome particles within a dispersion solution. As used herein, the “water-soluble drugs” are those that 1 gram of solute is soluble in less than 1000 mL of water at 25° C. Preferably, the water-soluble drug is an anti-tumor drug. As used herein, the “empty-liposomes” are those liposomes not encapsulating any drug.

The combined use of the saccharide and the cyclodextrin or cyclodextrin derivative as the lyoprotectant, and particularly the defined ratio of the saccharide to the cyclodextrin or cyclodextrin derivative, lead to a synergistic effect between the two lyoprotectants of the composition, and have a noticeable positive effect.

Furthermore, extensive experiments conducted by inventors of the present invention ensured that the methods fulfill the purpose of the present invention; following the step of preparation of empty-liposomes E, the addition of saccharide and cyclodextrin or cyclodextrin derivative into the system allows said two lyoprotectants to be positioned in the outer phase of the liposomes F encapsulating the water-soluble drug. This step allows said lyoprotectants to function in their role of protecting the structure of resultant liposome product, hence fulfilling the purpose of this invention. By using the saccharide and the cyclodextrin or the cyclodextrin derivative as the lyoprotectants for the liposome, changes in encapsulation rate and diameter of the liposome composition product before and after the lyophilization are minimized, allowing the product to be stored in a stable manner for over 24 months.

Preferably, the organic solvent used in said preparation processes is one or more selected from the group consisting of chloroform, ethanol, isopropanol and methanol.

Preferably, the first buffer used in said preparation processes is an ammounium salt solution, and the ammonium salt is one or more selected from the group consisting of ammonium phosphate, ammonium carbonate, ammonium chloride, ammonium sulfate and ammonium acetate.

Preferably, the second buffer used in said preparation processes is one or more selected from the group consisting of phosphate solution, sodium citrate solution, citric acid solution and histidine solution, wherein said phosphate is one or more selected from the group consisting of dipotassium hydrogen phosphate, potassium dihydrogen phosphate, disodium hydrogen phosphate and sodium dihydrogen phosphate.

A liposome dispersion-based drug delivery system is significantly advantageous in a clinical setting; the system can control drug distribution in the body, lower drug-plasma concentration, and decrease toxicity. However, the short shelf life of the system is detrimental to drug logistical distribution and usage. In order to promote the application of liposome technology, the inventors of the present invention have conducted extensive researches on liposome lyoprotectants and preparation methods thereof. Surprisingly, it is found that the combined use of the saccharide and the cyclodextrin or cyclodextrin derivative as a lyoprotectant can prevent the damage to the liposomes in a liposome dispersion during the lyophilization process, and this leads to the production of liposomes with minimal changes in particle diameter, whilst maintaining a high encapsulating rate. The lyophilized liposome composition, which is prepared by using the saccharide and cyclodextrin or cyclodextrin derivative as the lyoprotectant for lyophilization of the liposome dispersion, can maintain its encapsulation rate and liposome particle diameter with minimal change after redissolution at 0, 6, 9, 12, 18 or 24 months from manufacture date. Therefore, the lyophilized liposome composition encapsulating a water-soluble drug of the present invention can be stored for a long period.

The beneficial effects of the present invention are as follows: the saccharide and the cyclodextrin or cyclodextrin derivative that are used as the lyoprotectant composition for the preparation of a lyophilized liposome composition encapsulating a water-soluble drug, prevents the leakage of water-soluble dugs from liposomes, which leakage was due to deformation or damage of the liposomes during the lyophilization. The methods of the present invention increase the lyophilized composition's stability and prolong its shelf life whilst securing the quality of the encapsulated water-soluble drug. The lyophilized liposome composition encapsulating a water-soluble drug of the present invention has an encapsulation rate ≧90%, has a particle diameter between 10-500 nm, has a shelf life ≧24 months and has performances meet all corresponding standards. The lyophilized liposome composition encapsulating a water-soluble drug provided by the present invention facilitates the use of liposome formulations that have been proved to be advantageous in clinical applications, laying a foundation for the further use of liposomes encapsulating water-soluble drugs.

### DETAILED DESCRIPTION OF THE PREFERRED EMBODIMENTS OF THE INVENTION

The details of the present invention will be more fully understood and appreciated from the following description, taken in conjunction with the embodiments. While the preferred embodiments of the invention will be described below, these embodiments are not limitations on the protection scope of the present invention.

### EXAMPLE 1

Ingredients: 0.2 g of doxorubicin hydrochloride as the water-soluble drug, 0.9 g of HSPC as the phospholipid, 0.4 g of DSPE-mPEG2000 as the polyethylene glycol-derivatizedphospholipid, 0.25 g of cholesterol, 14.0 g of sucrose as the saccharide, and 1.25 g of hydroxypropyl-β-cyclodextrin as the cyclodextrin or cyclodextrin derivative.

Preparation process comprises the steps of: dissolving the phospholipid, the polyethylene glycol-derivatizedphospholipid and cholesterol in 500 mL of ethanol (99.5%) as the organic solvent to obtain a clear solution A; adding 250 mL of a first buffer, ammonium sulfate solution (250 mM) into the clear solution A with continuous stirring under a 60° C. water bath, extruding the resultant product using a high pressure homogenizer to obtain empty-liposomes D with an average particle diameter approximately 90 nm; dialyzing the first buffer solution from the empty-liposomes D by adding 5000 mL of a second buffer solution, histidine solution (10 mM) to obtain empty-liposomes E; dissolving the water-soluble drug and sucrose in 200 ml of the second buffer, 10 mM histidine solution, to obtain a solution B; mixing the empty-liposomes E and the solution B, encapsulating at 40° C. for 12 minutes followed by cooling at room temperature to obtain liposomes F, which encapsulating the water-soluble drug in its inner phase; preparing aliquots of the liposomes F into 10 bottles after adding hydroxypropyl-β-cyclodextrin, lyophilizing the aliquots to obtain the lyophilized liposome composition encapsulating the water-soluble drug of the present invention.

Three batches of the product were prepared, and subsequently referred to as Example 1-1, Example 1-2, and Example 1-3.

### EXAMPLE 2

Ingredients: 0.2 g of daunorubicin hydrochloride, 1.2 g of HSPC, 0.6 g of DSPE-mPEG2000, 1.0 g of cholesterol, 13.0 g of sucrose, 2.5 g of hydroxypropyl-β-cyclodextrin.

Preparation process: Example lwas repeated, but the temperature of water bath was 76° C., and the encapsulation temperature was 105° C.

Three batches of the product were prepared, and subsequently referred to as Example 2-1, Example 2-2, and Example 2-3.

### EXAMPLE 3

Ingredients: 0.2 g of doxorubicin hydrochloride, 0.32 g of HSPC, 0.18 g of DSPE-mPEG2000, 0.2 g of cholesterol, 13.9 g of sucrose, 0.78 g of hydroxypropyl-α-cyclodextrin.

Preparation process: Example 1 was repeated, but the organic solvent was 1000 mL of methanol (99.5%), the second buffer was 250 mL of citrate solution (10 mM), the temperature of water bath was 65° C., and the encapsulation temperature was80° C.

Three batches of the product were prepared, and subsequently referred to as Example 3-1, Example 3-2, and Example 3-3.

### EXAMPLE 4

Ingredients: 0.2 g of doxorubicin hydrochloride, 0.7 g of HSPC, 0.8 g of DSPE-mPEG2000, 0.4 g of cholesterol, 6.7 g of D-galactose, 4.6 g of 2-hydroxypropyl-β-cyclodextrin.

Preparation process: Example lwas repeated, but the organic solvent was 500 mL of isopropanol (99.5%), the first buffer was 300 mL of ammonium acetate solution (400 mM), the temperature of water bath was 55° C., and the encapsulation temperature was 100° C.

Three batches of the product were prepared, and subsequently referred to as Example 4-1, Example 4-2, and Example 4-3.

### EXAMPLE 5

Ingredients: 0.16 g of daunorubicin hydrochloride, 1.5 g of DSPG, 0.18 g of DPPG-mPEG2000, 0.36 g of cholesterol, 10.0 g of D-glucose, 3.0 g of 2-hydroxypropyl-β-cyclodextrin.

Preparation process: Example lwas repeated, but the temperature of water bath was 57° C., and the encapsulation temperature was 115° C.

Three batches of the product were prepared, and subsequently referred to as Example 5-1, Example 5-2, and Example 5-3.

### EXAMPLE 6

Ingredients: 1.5 g of pirarubicin hydrochloride, 0.9 g of DOPC, 0.4 g of HSPC-mPEG2000, 0.6 g of cholesterol, 9.6 g of maltose, 2.0 g of 2-hydroxypropyl-β-cyclodextrin.

Preparation process: Example 1 was repeated, but the organic solvent was 1000 mL of methanol, the first buffer was 200 mL of ammonium acetate solution (350 mM), the second buffer was 350 mL of potassium dihydrogen phosphate (5 mM), the temperature of water bath was 52° C., and the encapsulation temperature was 90° C.

Three batches of the product were prepared, and subsequently referred to as Example 6-1, Example 6-2, and Example 6-3.

### EXAMPLE 7

Ingredients: 0.08 g of epirubicin hydrochloride, 0.2 g of DPPG, 0.18 g of DOPC-mPEG2000, 0.2 g of cholesterol, 10.0 g of maltose, 4.0 g of 2-hydroxypropyl-β-cyclodextrin.

Preparation process: Example lwas repeated, but the organic solvent was 600 mL of isopropanol (99.7%), the second buffer was 250 mL ofcitrate solution (8 mM), the temperature of water bath was 63° C., and the encapsulation temperature was 112° C.

Three batches of the product were prepared, and subsequently referred to as Example 7-1, Example 7-2, and Example 7-3.

### EXAMPLE 8

Ingredients: 0.2 g of doxorubicin hydrochloride, 0.9 g of HSPC, 0.4 g of DSPE-mPEG2000, 0.25 g of cholesterol, 9.0 g of D-mannitol, 2.0 g of hydroxypropyl-γ-cyclodextrin.

Preparation process: Example 1 was repeated, but the organic solvent was 100 mL of chloroform (99.0%), the first buffer was ammonium acetate solution (300 mM), the second buffer was 230 mL of sodium phosphate (10 mM), the temperature of water bath was 50° C., and the encapsulation temperature was 60° C.

Three batches of the product were prepared, and subsequently referred to as Example 8-1, Example 8-2, and Example 8-3.

### EXAMPLE 9

Ingredients: 0.1 g of pirarubicin hydrochloride, 0.5 g of HSPC, 0.9 g of HSPC-mPEG2000, 1.0 g of cholesterol, 10.0 g of D-mannitol, 3.0 g of hydroxypropyl-β-cyclodextrin.

Preparation process: Example 1 was repeated, but the organic solvent was 180 mL of chloroform (99.0%), the first buffer was 300 mL of ammonium acetate solution (200 mM), the temperature of water bath was 75° C., and the encapsulation temperature was 98° C.

Three batches of the product were prepared, and subsequently referred to as Example 9-1, Example 9-2, and Example9-3.

### EXAMPLE 10

Ingredients: 0.1 g of doxorubicin hydrochloride, 0.7 g of DOPC, 1.36 g of DSPE-mPEG2000, 1.09 g of cholesterol, 8.65 g of sucrose, 1.75 g of hydroxypropyl-γ-cyclodextrin.

Preparation process: Example lwas repeated, but the temperature of water bath was 68° C., and the encapsulation temperature was90° C.

Three batches of the product were prepared, and subsequently referred to as Example 10-1, Example 10-2, and Example 10-3.

### EXAMPLE 11

Ingredients: 1.0 g of pirarubicin hydrochloride, 0.9 g of HSPC, 0.5 g of DSPE-mPEG2000, 0.25 g of cholesterol, 11.5 g of D-glucose, 2 g of α-cyclodextrin.

Preparation process: Example lwas repeated, but the temperature of water bath was 63° C., and the encapsulation temperature was 80° C.

Three batches of the product were prepared, and subsequently referred to as Example 11-1, Example 11-2, and Example 11-3.

### EXAMPLE 12

Ingredients: the ingredients and the amounts thereof were the same as those in Example 1.

Preparation process comprises the steps of: dissolving the phospholipid, the polyethylene glycol-derivatized phospholipid and cholesterol in isopropanol as the organic solvent to obtain a clear solution A; adding the ammonium sulfate solution as a first buffer into the clear solution A with continuous stirring under a 50° C. water bath, and extruding the resultant product to obtain empty-liposomes D with an average particle diameter from approximately 10 to 500 nm; dialyzing the first buffer solution (the ammonium sulfate solution) from the empty-liposomes D by adding the histidine solution as a second buffer solution to obtain empty-liposomes E; dissolving the water-soluble drug in the second buffer, the histidine solution, to obtain a solution B; mixing the solution B with the empty-liposomes E, encapsulating at 40° C. for 15 minutes followed by cooling at room temperature to obtain liposomes F encapsulating the water-soluble drug; adding the two lyoprotectants into the liposomes F, then preparing aliquots of the resultant product into 10 bottles, lyophilizing to obtain the lyophilized liposome composition encapsulating the water-soluble drug of the present invention.

Three batches of the product were prepared, and subsequently referred to as Example 12-1, Example 12-2, and Example 12-3.

### EXAMPLE 13

Ingredients: the ingredients and the amounts thereof were the same as those in Example 1.

Preparation process: Example 1 was repeated, but the encapsulation temperature was 60° C., and the encapsulation time was 40 minutes.

Three batches of the product were prepared, and subsequently referred to as Example 13-1, Example 13-2, and Example 13-3.

### EXAMPLE 14

Ingredients: the ingredients and the amounts thereof were the same as those in Example 1.

Preparation process: Example 1 was repeated, but the encapsulation time was 60 minutes.

Three batches of the product were prepared, and subsequently referred to as Example 14-1, Example 14-2, and Example 14-3.

### EXAMPLE 15

Ingredients: the ingredients and the amounts thereof were the same as those in Example 1.

Preparation process: Example 1 was repeated, but the encapsulation temperature was 80° C., and the encapsulation time was 8 minutes.

Three batches of the product were prepared, and subsequently referred to as Example 15-1, Example 15-2, and Example 15-3.

### EXAMPLE 16

Ingredients: the ingredients and the amounts thereof were the same as those in Example 1.

Preparation process: Example 1 was repeated, but the encapsulation temperature was 70° C., and the encapsulation time was 45 minutes.

Three batches of the product were prepared, and subsequently referred to as Example 16-1, Example 16-2, and Example 16-3.

### COMPARATIVE EXAMPLE 1

Commercially available liposomal doxorubicin hydrochloride injection (Doxil, manufactured by Ben Venue Laboratories, Inc., Batch No. 071844224, shelf life: 20 months).

### COMPARATIVE EXAMPLE 2

Ingredients: the ingredients and the amounts thereof were the same as those in Example 1, but neither the cyclodextrin nor the cyclodextrin derivative was added to the outer phase of the liposome sencapsulating the water-soluble drug.

Preparation process: Example 1 was repeated, but hydroxypropyl-β-cyclodextrin was added to the liposomes F. The resultant product was subsequently referred to as Comparison 2.

### COMPARATIVE EXAMPLE 3

Ingredients: the ingredients and the amounts thereof were the same as those in Example 1, but sucrose was not added to the outer phase of the empty liposomes.

Preparation process: Example 1 was repeated, but sucrose was not dissolved in the second buffer, the histidine solution. The resultant product was subsequently referred to as Comparison 3.

Parameters regarding quality, stability and shelf life were measured on the lyophilized compositions from Example 1-16 and Comparative example 2 and 3, through methods that are well known to one skilled in the art.

I. Encapsulation rate and average liposome particle diameter were measured on pre-lyophilized liposome compositions from Example 1-16 and Comparative example 2 and 3, as well as on their corresponding redissolved lyophilized compositions.

TABLE 1
Encapsulation rate (%) and average liposome particle diameter (nm)
measured on pre-lyophilized liposomal compositions from Example
1-16 and Comparative example 2 and 3, as well as on
corresponding redissolved lyophilized compositions

| | | | 0 hour after redissolution | |
|---|---|---|---|---|
| | | | of the lyophilized | |
| | Before lyophilization | | liposome compositions | |
| | | Average | | Average |
| | | Particle | Encapsulation | Particle |
| | Encapsulation | Diameter | Rate | Diameter |
| Sample | Rate (%) | (nm) | (%) | (nm) |
| Example 1-1 | 97.6 | 78.5 | 96.5 | 81.0 |
| Example1-2 | 97.8 | 78.9 | 96.3 | 82.2 |
| Example 1-3 | 98.5 | 78.0 | 96.9 | 81.8 |
| Example2-1 | 96.9 | 82.5 | 95.4 | 85.0 |
| Example2-2 | 97.3 | 83.5 | 96.1 | 85.4 |
| Example2-3 | 96.8 | 83.0 | 95.5 | 85.3 |
| Example3-1 | 93.7 | 83.4 | 92.2 | 85.7 |
| Example4-1 | 95.3 | 82.6 | 94.2 | 85.0 |
| Example5-1 | 93.1 | 130.6 | 90.8 | 137.5 |
| Example6-1 | 93.6 | 140.1 | 91.5 | 148.9 |
| Example7-1 | 95.1 | 100.5 | 90.1 | 110.3 |
| Example8-1 | 94.2 | 123.0 | 92.5 | 127.2 |
| Example 9-1 | 95.9 | 150.1 | 94.3 | 158.1 |
| Example 10-1 | 93.5 | 95.4 | 93.0 | 98.7 |
| Example 11-1 | 91.3 | 100.5 | 90.6 | 110.1 |
| Example 12-1 | 97.6 | 83.8 | 96.4 | 86.0 |
| Example 13-1 | 98.7 | 78.8 | 97.5 | 81.6 |
| Example 14-1 | 98.2 | 78.3 | 97.1 | 81.2 |
| Example 15-1 | 95.5 | 79.0 | 94.7 | 82.4 |
| Example 16-1 | 99.0 | 78.4 | 98.3 | 81.1 |
| Comparison2 | 95.6 | 110.6 | 60.5 | 120.4 |
| Comparison 3 | 88.4 | 84.3 | 50.6 | 163.2 |

As shown in Table 1, the lyophilized liposome compositions from Examples 1-16 of the present invention remain largely unchanged after redissolving from its lyophilized form, as compared to their corresponding pre-lyophilized compositions in terms of encapsulation rate and liposome particle diameter.

The tested sample Comparison 2, containing a saccharide, without containing cyclodextrin or cyclodextrin derivative, only had an encapsulation rate of 60.5% after redissolving, lower than the required 80% for liposomal drugs, and far lower than its pre-lyophilization encapsulation rate (95.6%).

For the tested sample Comparison 3, which contains a cyclodextrin or cyclodextrin derivative, without containing saccharide, there was a significant difference between the pre-lyophilized compositions and the post-lyophilized compositions in terms of encapsulation rate and liposome particle diameter. Before the lyophilization, the encapsulation rate was 88.4%, afterwards, it dropped to 50.6%, whilst the particle diameter increased from 84.3 to 163.2 nm.

It can be seen from above results that, the combined use of the saccharide and the cyclodextrin or cyclodextrin derivative can prevent the liposomal drugs from deformation or being damaged during the lyophilization process, hence avoiding large-scale leakage of the encapsulated core material from liposomes. The lyophilized liposome composition provided by the present invention can achieve an encapsulation rate of over 90%, and a liposome particle diameter between 50-500 nm both before and after the lyophilization. Most importantly, there is no significant change in the encapsulation rate and the particle diameter before and after the lyophilization.

### II. Stability Analysis

An accelerated stability study was conducted upon the lyophilized liposome composition from Examples 1-16, along with the product from Comparative example 1. Products from Comparative example 2 and 3 were excluded in this study, because they could not meet the requirements for liposomal drugs after the lyophilization process.

(1) Stability study conducted on compositions stored in an environment having a temperature of 25° C.: lyophilized liposome compositions from Examples 1-16, and the commercially available product from Comparative example 1 were stored in a 25° C. environment for 0, 1, 2, 3 and 6 months; these samples were then tested for the weight percent of the water-soluble drug in the composition and weight percent of related substances in the composition, for the purpose of studying the changes of the concentration of water-soluble drug during the storage, the results are listed in Table 2.

TABLE 2
Experimental results of the accelerated stability study (storage
temperature 25° C.) measured on lyophilized liposome compositions from
Examples 1-16 and on the product from Comparative Example 1

| | 0 month | | 1 month | | 2 months | | 3 months | | 6 months | |
|---|---|---|---|---|---|---|---|---|---|---|
| | Water- | | Water- | | Water- | | Water- | | Water- | |
| | soluble | Related | soluble | Related | soluble | Related | sobible | Related | soluble | Related |
| | drug | substances | drug | substances | drug | substances | drug | substances | drug | substances |
| Comparative | 99.1% | 1.11% | 99.5% | 1.52% | 99.7% | 2.42% | 96.0% | 3.57% | 91.9% | 8.04% |
| Example 1 | | | | | | | | | | |
| Example | 100% | 0.22% | 99.8% | 0.70% | 99.6% | 1.03% | 98.2% | 1.60% | 96.1% | 2.30% |
| 1-1 | | | | | | | | | | |
| Example | 100% | 0.25% | 99.7% | 0.72% | 99.3% | 1.04% | 98.0% | 1.64% | 96.8% | 2.35% |
| 1-2 | | | | | | | | | | |
| Example | 99.7% | 0.23% | 99.4% | 0.71% | 98.9% | 1.12% | 98.5% | 1.69% | 97.5% | 2.30% |
| 1-3 | | | | | | | | | | |
| Example | 99.8% | 0.31% | 99.3% | 0.75% | 98.5% | 1.15% | 98.3% | 1.74% | 97.3% | 2.51% |
| 2-1 | | | | | | | | | | |
| Example | 99.5% | 0.30% | 99.6% | 0.74% | 98.9% | 1.17% | 98.0% | 1.78% | 97.9% | 2.59% |
| 2-2 | | | | | | | | | | |
| Example | 99.7% | 0.31% | 99.6% | 0.73% | 99.0% | 1.12% | 98.4% | 1.69% | 97.5% | 2.48% |
| 2-3 | | | | | | | | | | |
| Example | 94.0% | 0.26% | 93.86% | 0.70% | 93.6% | 1.00% | 92.5% | 1.63% | 91.9% | 2.43% |
| 3-1 | | | | | | | | | | |
| Example | 97.8% | 0.34% | 97.4% | 0.78% | 97.0% | 1.20% | 95.8% | 1.80% | 94.0% | 2.53% |
| 4-1 | | | | | | | | | | |
| Example | 96.7% | 0.30% | 96.4% | 0.73% | 96.2% | 1.15% | 95.7% | 1.74% | 94.0% | 2.48% |
| 5-1 | | | | | | | | | | |
| Example | 98.9% | 0.21% | 98.7% | 0.69% | 98.5% | 1.00% | 97.1% | 1.59% | 96.0% | 2.28% |
| 6-1 | | | | | | | | | | |
| Example | 95.9% | 0.24% | 95.6% | 0.71% | 95.2% | 1.01% | 93.9% | 1.63% | 93.3% | 2.33% |
| 7-1 | | | | | | | | | | |
| Example | 96.6% | 0.22% | 96.3% | 0.70% | 96.0% | 1.09% | 95.2% | 1.68% | 94.8% | 2.28% |
| 8-1 | | | | | | | | | | |
| Example | 97.7% | 0.30% | 97.5% | 0.74% | 97.3% | 1.12% | 96.4% | 1.73% | 95.0% | 2.49% |
| 9-1 | | | | | | | | | | |
| Example | 97.4% | 0.29% | 97.3% | 0.73% | 97.1% | 1.14% | 96.5% | 1.77% | 95.4% | 2.57% |
| 10-1 | | | | | | | | | | |
| Example | 95.6% | 0.30% | 95.4% | 0.72% | 95.3% | 1.09% | 95.0% | 1.68% | 94.2% | 2.46% |
| 11-1 | | | | | | | | | | |
| Example | 100% | 0.24% | 99.7% | 0.75% | 99.6% | 1.08% | 98.2% | 1.64% | 96.1% | 2.35% |
| 12-1 | | | | | | | | | | |
| Example | 100% | 0.22% | 99.8% | 0.70% | 99.7% | 1.02% | 99.0% | 1.63% | 96.5% | 2.36% |
| 13-1 | | | | | | | | | | |
| Example | 99.9% | 0.21% | 99.6% | 0.72% | 99.6% | 1.06% | 98.7% | 1.62% | 96.1% | 2.39% |
| 14-1 | | | | | | | | | | |
| Example | 99.8% | 0.25% | 99.5% | 0.75% | 99.3% | 1.09% | 98.6% | 1.67% | 95.9% | 2.42% |
| 15-1 | | | | | | | | | | |
| Example | 100% | 0.22% | 99.7% | 0.70% | 99.5% | 1.04% | 98.8% | 1.62% | 96.4% | 2.34% |
| 16-1 | | | | | | | | | | |

As shown in Table 2, for the comparative product, the weight percent of water-soluble drug in the product significantly decreased after 6 months of storage at 25° C., while the weight percent of related substances significantly increased. However, the weight percent of water-soluble drug and related substances in the lyophilized liposome composition prepared according to Examples 1-16 of the present invention experienced relatively smaller changes over the same period, satisfying quality requirements for liposomal drugs. Hence, the results indicate that the lyophilized liposome composition encapsulating the water-soluble drug of the present invention shows an improved long-term stability as compared to the product provided by Comparative Example 1.

(2) Stability study conducted on compositions stored in an environment having a low temperature between 2-10 ° C.: lyophilized liposome compositions from Examples 1-16, and the commercially available product from Comparative Example 1 were stored in an environment having a low temperature between 2-10 ° C. for 0, 6, 9, 12, 20 and 24 months, and then the following parameters were tested:

1) encapsulation rate (%) of the redissolved lyophilized liposome products after 0, 6, 9, 12, 20, 24 months of storage (Table 3);

2) liposome particle diameter (nm) of the redissolved lyophilized liposome products after 0, 6, 9, 12, 20, 24 months of storage (Table 4);

3) weight percent of water-soluble drug (%) and related substances (%)in the redissolved lyophilized liposome products after 0, 6, 9, 12, 20, 24 months of storage (Table 5);

4) release rate (%) of the lyophilized liposome composition after 0, 6, 9, 12, 20, 24 months of storage (Table 6).

TABLE 3
Encapsulation rate (%) measured on redissolved lyophilized liposome
products prepared from Examples 1-16 and on the product of
Comparative Example 1, after a long-term cold (2-10° C.) storage

| | 0 | 6 | 9 | 12 | 20 | 24 |
|---|---|---|---|---|---|---|
| | month | months | months | months | months | months |
| Comparative | 98.0 | 97.8 | 98.0 | 97.5 | 97.0 | 96.5 |
| Example 1 | | | | | | |
| Example 1-1 | 96.5 | 96.5 | 96.6 | 96.5 | 96.8 | 95.8 |
| Example 1-2 | 96.3 | 96.8 | 96.8 | 96.5 | 96.6 | 96.8 |
| Example 1-3 | 96.9 | 97.0 | 97.2 | 96.1 | 96.5 | 97.1 |
| Example 2-1 | 95.4 | 95.7 | 95.5 | 95.5 | 95.3 | 95.5 |
| Example 2-2 | 96.1 | 96.5 | 96.3 | 96.2 | 96.4 | 96.5 |
| Example 2-3 | 95.5 | 95.4 | 95.5 | 95.3 | 95.5 | 95.5 |
| Example 3-1 | 92.2 | 92.5 | 92.4 | 92.4 | 92.4 | 92.3 |
| Example 4-1 | 94.2 | 94.4 | 94.6 | 94.6 | 94.2 | 94.4 |
| Example 5-1 | 90.8 | 90.0 | 90.3 | 90.9 | 90.5 | 90.1 |
| Example 6-1 | 91.5 | 91.6 | 91.7 | 91.7 | 91.7 | 91.7 |
| Example 7-1 | 90.1 | 90.8 | 90.9 | 90.9 | 90.9 | 90.9 |
| Example 8-1 | 92.5 | 92.7 | 92.8 | 92.8 | 92.8 | 92.8 |
| Example 9-1 | 94.3 | 94.1 | 94.2 | 94.2 | 94.2 | 94.2 |
| Example 10-1 | 93.0 | 93.0 | 93.1 | 93.1 | 93.1 | 93.1 |
| Example 11-1 | 90.6 | 90.8 | 90.9 | 90.9 | 90.9 | 90.9 |
| Example 12-1 | 96.4 | 96.5 | 96.6 | 96.5 | 96.8 | 95.8 |
| Example 13-1 | 97.5 | 97.5 | 97.4 | 97.2 | 97.2 | 97.1 |
| Example 14-1 | 97.1 | 97.0 | 97.1 | 96.9 | 96.7 | 96.5 |
| Example 15-1 | 94.7 | 94.7 | 94.5 | 94.2 | 94.1 | 93.8 |
| Example 16-1 | 98.3 | 98.4 | 98.3 | 98.0 | 98.1 | 97.8 |

As shown in Table 3, there was no significant change in the encapsulation rate after long-term, low-temperature storage for either the product of Comparative Example 1 or the redissolved lyophilized liposome products prepared from Examples 1-16 of the present invention.

TABLE 4
Liposome particle diameter (nm) measured on redissolved lyophilized
liposome products prepared from Examples 1-16 and the product of
Comparative Example 1, after a long-term cold (2-10° C.) storage

| | 0 | 6 | 9 | 12 | 20 | 24 |
|---|---|---|---|---|---|---|
| | month | months | months | months | months | months |
| Comparative | 81.5 | 81.4 | 82.9 | 83.4 | 85.8 | 88.0 |
| Example 1 | | | | | | |
| Example 1-1 | 81.0 | 81.0 | 82.3 | 82.6 | 82.7 | 83.0 |
| Example 1-2 | 82.2 | 82.0 | 82.3 | 82.3 | 82.8 | 83.4 |
| Example 1-3 | 81.8 | 81.8 | 82.1 | 82.5 | 82.8 | 83.0 |
| Example 2-1 | 85.0 | 84.6 | 84.7 | 84.5 | 85.3 | 84.6 |
| Example 2-2 | 85.4 | 85.0 | 85.2 | 85.9 | 85.0 | 85.2 |
| Example 2-3 | 85.3 | 85.4 | 85.2 | 85.9 | 84.9 | 85.5 |
| Example 3-1 | 85.7 | 85.6 | 85.5 | 86.3 | 86.8 | 86.0 |
| Example 4-1 | 85.0 | 85.8 | 85.8 | 85.7 | 85.6 | 85.6 |
| Example 5-1 | 137.5 | 137.0 | 139.5 | 139.4 | 139.2 | 139.8 |
| Example 6-1 | 148.9 | 144.3 | 150.5 | 149.4 | 149.7 | 150.6 |
| Example 7-1 | 110.3 | 111.0 | 113.0 | 109.0 | 116.0 | 114.9 |
| Example 8-1 | 127.2 | 123.5 | 124.5 | 125.5 | 125.5 | 129.4 |
| Example 9-1 | 158.1 | 159.4 | 159.0 | 161.4 | 161.8 | 162.0 |
| Example 10-1 | 98.7 | 96.7 | 96.7 | 98.7 | 97.7 | 98.6 |
| Example 11-1 | 110.1 | 112.1 | 112.9 | 113.5 | 113.8 | 114.0 |
| Example 12-1 | 86.0 | 85.0 | 85.0 | 85.0 | 85.0 | 84.9 |
| Example 13-1 | 81.6 | 81.9 | 81.9 | 82.1 | 82.4 | 82.5 |
| Example 14-1 | 81.2 | 81.7 | 81.8 | 81.9 | 82.2 | 82.4 |
| Example 15-1 | 82.4 | 82.7 | 82.6 | 82.8 | 83.5 | 83.5 |
| Example 16-1 | 81.1 | 81.3 | 81.5 | 81.5 | 81.9 | 81.9 |

As shown in Table 4, there was no significant change in the liposome particle diameter after long-term, low-temperature storage for either the product of Comparative Example 1 or the redissolved lyophilized liposome products prepared from Examples 1-16 of the present invention.

TABLE 5
Weight percent of water-soluble drug (%) and related substances (%)
measured on redissolved lyophilized liposome products prepared from Examples
1-16 and the product of Comparative Example1 after a long-term cold (2-10° C.)
storage

| | 0 month | | 6 months | | 12 months | | 20 months | | 24 months | |
|---|---|---|---|---|---|---|---|---|---|---|
| | Water- | | Water- | | Water- | | Water- | | Water- | |
| | soluble | Related | soluble | Related | soluble | Related | soluble | Related | soluble | Related |
| | drug | Substances | drug | Substances | drug | Substances | drug | Substances | drug | Substances |
| Comparative | 99.1% | 1.11% | 98.6% | 1.5% | 98.2% | 2.1% | 96.8% | 2.3% | 95.9 | 3.0% |
| Example 1 | | | | | | | | | | |
| Example 1-1 | 100% | 0.22% | 99.6% | 0.21% | 99.6% | 0.30% | 99.7% | 0.36% | 100% | 0.39% |
| Example 1-2 | 100% | 0.25% | 99.9% | 0.19% | 99.8% | 0.25% | 99.7% | 0.35% | 99.5% | 0.39% |
| Example 1-3 | 99.7% | 0.23% | 99.8% | 0.20% | 99.7% | 0.30% | 99.6% | 0.36% | 99.7% | 0.40% |
| Example 2-1 | 99.8% | 0.31% | 99.5% | 0.24% | 99.5% | 0.32% | 99.6% | 0.36% | 99.4% | 0.40% |
| Example 2-2 | 99.5% | 0.30% | 99.5% | 0.23% | 99.3% | 0.25% | 99.4% | 0.31% | 99.5% | 0.37% |
| Example 2-3 | 99.7% | 0.31% | 99.5% | 0.26% | 99.6% | 0.29% | 99.1% | 0.30% | 99.5% | 0.36% |
| Example 3-1 | 94.0% | 0.26% | 94.1% | 0.26% | 94.0% | 0.29% | 93.8% | 0.30% | 94.0% | 0.35% |
| Example 4-1 | 97.8% | 0.34% | 97.8% | 0.25% | 97.6% | 0.28% | 97.5% | 0.3% | 97.5% | 0.38% |
| Example 5-1 | 96.7% | 0.30% | 96.7% | 0.26% | 96.4% | 0.30% | 96.6% | 0.35% | 96.5% | 0.40% |
| Example 6-1 | 98.9% | 0.21% | 98.7% | 0.22% | 98.7% | 0.31% | 98.6% | 0.37% | 98.4% | 0.41% |
| Example 7-1 | 95.9% | 0.24% | 95.9% | 0.26% | 95.6% | 0.33% | 95.5% | 0.37% | 95.6% | 0.41% |
| Example 8-1 | 96.6% | 0.22% | 96.6% | 0.25% | 96.4% | 0.26% | 96.5% | 0.32% | 96.3% | 0.38% |
| Example 9-1 | 97.7% | 0.30% | 97.6% | 0.28% | 97.2% | 0.30% | 97.3% | 0.31% | 97.4% | 0.37% |
| Example | 97.4% | 0.29% | 97.6% | 0.28% | 97.5% | 0.30% | 97.0% | 0.31% | 97.4% | 0.36% |
| 10-1 | | | | | | | | | | |
| Example | 95.6% | 0.30% | 95.3% | 0.27% | 95.9% | 0.29% | 95.4% | 0.31% | 95.2% | 0.39% |
| 11-1 | | | | | | | | | | |
| Example | 100% | 0.25% | 99.9% | 0.19% | 99.8% | 0.25% | 99.7% | 0.35% | 99.5% | 0.39% |
| 12-1 | | | | | | | | | | |
| Example | 100% | 0.22% | 99.7% | 0.21% | 99.7% | 0.28% | 99.6% | 0.33% | 99.6% | 0.38% |
| 13-1 | | | | | | | | | | |
| Example | 99.9% | 0.21% | 99.7% | 0.22% | 99.6% | 0.26% | 99.6% | 0.33% | 99.4% | 0.35% |
| 14-1 | | | | | | | | | | |
| Example | 99.8% | 0.25% | 99.5% | 0.24% | 99.3% | 0.29% | 99.2% | 0.36% | 99.3% | 0.42% |
| 15-1 | | | | | | | | | | |
| Example | 100% | 0.22% | 99.8% | 0.20% | 99.8% | 0.25% | 99.8% | 0.32% | 99.8% | 0.36% |
| 16-1 | | | | | | | | | | |

As shown in Table 5, there was no significant change in the weight percent of water-soluble drug and related substances after long-term, low-temperature storage for either the product of Comparative Example 1 or the redissolved lyophilized liposome products prepared from Examples 1-16 of the present invention. However, the products from Examples 1-16 of the present invention has a greater level of stability and an improved homogeneity than the product of Comparative Example 1.

TABLE 6
Release rate (%) measured on redissolved lyophilized liposome products
prepared from Examples 1-16 and the product of Comparative Example 1,
after a long-term cold (2-10° C.) storage

| | 0 month | | 6 months | | 12 months | | 20 months | | 24 months | |
|---|---|---|---|---|---|---|---|---|---|---|
| | 0.5 h | 3 h | 0.5 h | 3 h | 0.5 h | 3 h | 0.5 h | 3 h | 0.5 h | 3 h |
| Comparative | 66 | 82 | 66 | 82 | 68 | 83 | 69 | 83 | 74 | 84 |
| Example 1 | | | | | | | | | | |
| Example 1-1 | 60 | 81 | 61 | 81 | 61 | 82 | 62 | 83 | 62 | 82 |
| Example 1-2 | 61 | 80 | 61 | 80 | 62 | 80 | 62 | 82 | 63 | 82 |
| Example 1-3 | 63 | 82 | 63 | 82 | 63 | 83 | 63 | 82 | 63 | 82 |
| Example 2-1 | 62 | 81 | 62 | 81 | 62 | 83 | 63 | 83 | 63 | 83 |
| Example 2-2 | 61 | 82 | 62 | 82 | 62 | 83 | 62 | 83 | 62 | 83 |
| Example 2-3 | 63 | 80 | 63 | 80 | 63 | 81 | 63 | 81 | 63 | 81 |
| Example 3-1 | 62 | 80 | 62 | 80 | 63 | 81 | 63 | 81 | 63 | 82 |
| Example 4-1 | 62 | 81 | 63 | 81 | 63 | 81 | 63 | 82 | 63 | 84 |
| Example 5-1 | 63 | 80 | 63 | 80 | 63 | 81 | 63 | 81 | 63 | 81 |
| Example 6-1 | 60 | 79 | 60 | 79 | 61 | 79 | 61 | 81 | 62 | 81 |
| Example 7-1 | 62 | 81 | 62 | 81 | 62 | 82 | 62 | 81 | 62 | 81 |
| Example 8-1 | 61 | 80 | 61 | 80 | 61 | 82 | 62 | 82 | 62 | 82 |
| Example 9-1 | 60 | 81 | 61 | 81 | 61 | 82 | 61 | 82 | 61 | 82 |
| Example 10-1 | 62 | 79 | 62 | 79 | 62 | 80 | 62 | 80 | 62 | 80 |
| Example 11-1 | 61 | 79 | 61 | 79 | 62 | 80 | 62 | 80 | 62 | 81 |
| Example 12-1 | 61 | 80 | 61 | 80 | 62 | 80 | 62 | 82 | 63 | 82 |
| Example 13-1 | 60 | 80 | 61 | 82 | 61 | 83 | 61 | 83 | 62 | 82 |
| Example 14-1 | 61 | 79 | 61 | 80 | 63 | 82 | 62 | 82 | 62 | 83 |
| Example 15-1 | 63 | 82 | 62 | 80 | 62 | 81 | 62 | 81 | 64 | 83 |
| Example 16-1 | 61 | 81 | 61 | 81 | 62 | 82 | 62 | 83 | 63 | 81 |

As shown in Table 6, the release rate of the product provided by Comparative Example 1 failed to satisfy corresponding quality standards at 24 months; in the quality standards, the release rate within 0.5 hours should not exceed 70%, which might explains the shelf life of only 20 months for the product provided by Comparative Example 1. In contrast, the change in release rate of the products of the present invention was not significant after 24 months of storage, which meets the requirement of the standards.

In summary, a saccharide and a cyclodextrin or cyclodextrin derivative are combined as a lyoprotectant for the preparation of the lyophilized liposome composition encapsulating a water-soluble drug of the present invention. Preferably, the saccharide is added to the outer phase of an empty-liposome dispersion or a liposome dispersion with the liposome encapsulating water-soluble drugs, while the cyclodextrin is added to the outer phase of a liposome dispersion with the liposome encapsulating water-soluble drugs. In this way, both the saccharide and the cyclodextrin or the cyclodextrin derivative, are now combined as a lyoprotectant for liposomal drugs. Said lyoprotectant can be successfully applied to the lyophilization of a liposomal dispersion containing a water-soluble drug, with inapparent changes in encapsulation rate and liposome particle diameter for the liposomal drug before and after the lyophilization. Besides, the lyophilized liposome composition of the present invention has inapparent changes in various quality parameters after stored at a low temperature for 24 months, which meets the requirements in the standard. The lyophilized liposome composition encapsulating a water-soluble drug of the present invention can be stably stored for over 24 months, which is a marked progress compared to existing liposomes encapsulating water-soluble drugs, such as the product from Comparative Example 1, which has a shelf life of 18-20 months. Thus, the compositions of the present invention have obvious progresses as compared with existing ones, and are very useful in clinical applications.

It is intended that the foregoing detailed description be regarded as illustrative rather than limiting, and that it be understood that it is the following claims, including all equivalents, that are intended to define the spirit and scope of this invention.

1. A lyoprotectant composition used for the preparation of a liposome encapsulating a water-soluble drug, comprising
(a) a saccharide, and
(b) a cyclodextrin or cyclodextrin derivative.

2. The lyoprotectant composition as claimed in claim 1, wherein the mass ratio of the saccharide to the cyclodextrin or cyclodextrin derivative is 50-90:5-35.

3. A lyophilized liposome composition encapsulating a water-soluble drug, wherein the composition comprises the following ingredients: a water-soluble drug, a phospholipid, a polyethylene glycol-derivatized phospholipid, cholesterol, a saccharide and a cyclodextrin or cyclodextrin derivative.

4. The lyophilized liposome composition as claimed in claim 3, wherein the weight percent of each ingredient contained in the composition is as follows: 0.5-10% by weight of the water-soluble drug, 1-10% by weight of the phospholipid, 1-12% by weight of the polyethylene glycol-derivatized phospholipid, 1-15% by weight of the cholesterol, 50-90% by weight of the saccharide, and 5-35% by weight of the cyclodextrin or cyclodextrin derivative.

5. The lyophilized liposome composition as claimed in claim 4, wherein the weight percent of each ingredient contained in the composition is as follows: 0.5-10% by weight of the water-soluble drug, 1-10% by weight of the phospholipid, 1-12% by weight of the polyethylene glycol-derivatized phospholipid, 1-15% by weight of the cholesterol, 70-80% by weight of the saccharide, and 5-15% by weight of the cyclodextrin or cyclodextrin derivative.

6. The composition as claimed in claim, wherein,
the saccharide is one or more selected from the group consisting of D-glucose, D-galactose, D-mannitol, maltose, sucrose, trehalose and xylitol;
the cyclodextrin or the cyclodextrin derivative is one or more selected from the group consisting of hydroxypropyl-α-cyclodextrin, hydroxypropyl-β-cyclodextrin, 2-hydroxypropyl-β-cyclodextrin, hydroxypropyl-γ-cyclodextrin.

7. The lyophilized liposome composition as claimed in claim 4, wherein,
the phospholipid is one selected from the group consisting of egg lecithin, soya bean lecithin, distearoylphosphatidylglycerol (DSPG), hydrogenated soya phosphatidylcholine (HSPC), dioleoylphosphatidylcholine (DOPC), dipalmitoylphosphatidylglycerol (DPPG), distearoylphosphatidylethanolamine (DSPE);
the portion of phospholipid in the polyethylene glycol-derivatized phospholipid is one selected from the group consisting of distearoylphosphatidylglycerol (DSPG), hydrogenated soya phosphatidylcholine (HSPC), dioleoylphosphatidylcholine (DOPC), dipalmitoylphosphatidylglycerol (DPPG) and distearoylphosphatidylethanolamine (DSPE).

8. The lyophilized liposome composition as claimed in claim 7, wherein the polyethylene glycol of the polyethylene glycol-derivatized phospholipid has a molecular weight in the range from 2000 to 4000.

9. The lyophilized liposome composition as claimed in claim 8, wherein the polyethylene glycol of the polyethylene glycol-derivatized phospholipid has a molecular weight of 2000.

10. The lyophilized liposome composition as claimed in claim 4, wherein the water-soluble drug is one or more selected from the group consisting of doxorubicin, daunorubicin, epirubicin, pirarubicin, and pharmaceutically acceptable salts thereof.

11. The lyophilized liposome composition as claimed in claim 4, wherein the weight percent of each ingredient contained in the composition is as follows: 1-3% by weight of the doxorubicin hydrochloride, 5-8% by weight of the hydrogenated soya phosphatidylcholine (HSPC), 2-7% by weight of DSPE-mPEG2000 as the polyethylene glycol-derivatized phospholipid, 1-7% by weight of the cholesterol, 70-80% by weight of the sucrose and 5-15% by weight of the hydroxypropyl-β-cyclodextrin.

12. The lyophilized liposome composition as claimed in claim 4, wherein the weight percent of each ingredient contained in said composition is as follows: 1-3% by weight of the daunorubicin hydrochloride, 5-8% by weight of the hydrogenated soya phosphatidylcholine (HSPC), 2-7% by weight of the DSPE-mPEG2000 as the polyethylene glycol-derivatized phospholipid, 1-7% by weight of the cholesterol, 70-80% by weight of the sucrose and 5-15% by weight of the hydroxypropyl-β-cyclodextrin.

13. A process for preparing a lyophilized liposome composition according to claim 10, comprising the steps of:
(1) dissolving the phospholipid, the polyethylene glycol-derivatized phospholipid, and cholesterol in an organic solvent to obtain a clear solution A;
(2) adding a first buffer solution into the clear solution A with continuous stirring under a 50-80° C. water bath, extruding the resultant product to obtain empty-liposomes D with an average particle diameter from approximately 10 to 500 nm;
(3) dialyzing the first buffer solution from the empty-liposomes D by adding a second buffer solution to obtain empty-liposomes E;
(4) dissolving the water-soluble drug and the saccharide as a lyoprotectant in the second buffer to obtain a solution B;
(5) mixing the empty-liposomes E and the solution B, encapsulating at a temperature between 40-100° C. for 5-60 minutes followed by cooling at room temperature to obtain liposomes F, which encapsulating the water-soluble drug in its inner phase;
(6) preparing aliquots of the liposomes F after adding the cyclodextrin or cyclodextrin derivative as a lyoprotectant; lyophilizing the aliquots to obtain the lyophilized liposome composition encapsulating the water-soluble drug.

14. A process for preparing a lyophilized liposome composition according to claim 10, comprising the steps:
(1) dissolving the phospholipid, the polyethylene glycol-derivatized phospholipid and cholesterol in an organic solvent to obtain a clear solution A;
(2) adding a first buffer solution into the clear solution A with continuous stirring under a 50-80° C. water bath, extruding the resultant product to obtain empty-liposomes D with an average particle diameter from approximately 10 to 500 nm;
(3) dialyzing the first buffer solution from the empty-liposomes D by adding a second buffer solution to obtain empty-liposomes E;
(4) dissolving the water-soluble drug in the second buffer to obtain a solution B;
(5) mixing the empty-liposomes E and the solution B, encapsulating at a temperature between 40-100° C. for 5-60 minutes followed by cooling at room temperature to obtain liposomes F, with the inner phase of the liposomes F encapsulating the water-soluble drug;
(6) preparing aliquots of the liposomes F after adding the saccharide and the cyclodextrin or cyclodextrin derivative as a combined lyoprotectant; lyophilizing the aliquots to obtain the lyophilized liposome composition encapsulating the water-soluble drug.

15. The process as claimed in claim 13, wherein the organic solvent is one selected from the group consisting of chloroform, ethanol, isopropanol and methanol.

16. The process as claimed in claim 13, wherein,
the first buffer is an ammonium salt solution, and the ammonium salt is one or more selected from the group consisting of ammonium phosphate, ammonium carbonate, ammonium chloride, ammonium sulfate and ammonium acetate;
the second buffer is one or more selected from the group consisting of phosphate solution, sodium citrate solution, citric acid solution and histidine solution, wherein said phosphate is one or more selected from the group consisting of dipotassium hydrogen phosphate, potassium dihydrogen phosphate, disodium hydrogen phosphate and sodium dihydrogen phosphate.

17. The process as claimed in claim 14, wherein the organic solvent is one selected from the group consisting of chloroform, ethanol, isopropanol and methanol.

18. The process as claimed in claim 14, wherein,
the first buffer is an ammonium salt solution, and the ammonium salt is one or more selected from the group consisting of ammonium phosphate, ammonium carbonate, ammonium chloride, ammonium sulfate and ammonium acetate;
the second buffer is one or more selected from the group consisting of phosphate solution, sodium citrate solution, citric acid solution and histidine solution, wherein said phosphate is one or more selected from the group consisting of dipotassium hydrogen phosphate, potassium dihydrogen phosphate, disodium hydrogen phosphate and sodium dihydrogen phosphate.
